(19) United States
(12) Patent Application Publication
(10) Pub. No.: US 2026/0283818 A1
(43) Pub. Date: Sep. 24, 2026

(54) DEVICES AND INSTRUMENTS FOR SPINAL FUSION AND METHODS

(71) Applicant: Combination Spine, Inc., Bethesda, MD (US)

(72) Inventors: Joshua Michael Ammerman, Bethesda, MD (US); Spencer Austin Jones, Fayetteville, AR (US); Jordan R. Mykleby, Nashville, TN (US); James P. Duncan, Hernando, MS (US); Laurent Schaller, Genoa, NV (US)

(21) Appl. No.: 19/478,469

(22) PCT Filed: Apr. 26, 2024
(86) PCT No.: PCT/US2024/026421

§ 371 Date: Oct. 24, 2025

Related U.S. Application Data
(60) Provisional application No. 63/498,913, filed on Apr. 28, 2023.

(51) Int. Cl. A61F 2/44; A61F 2/30; A61F 2/46

(52) U.S. Cl. CPC A61F 2/446; A61F 2/30771; A61F 2/4611; A61F 2002/30125; A61F 2002/30476; A61F 2002/30523; A61F 2002/30556; A61F 2002/30579; A61F 2002/30622; A61F 2002/30845; A61F 2002/30904; A61F 2002/4622; A61F 2002/4627

(57) ABSTRACT

A device for treading vertebral disc spaces wherein the device includes a head with a body and rotating top and bottom blades. The device may include a deployment device for deploying the head into the disc space. The device may be a disc preparation device and/or a fusion implant.

The present application claims the benefit of and priority to U.S. Provisional Application No. 63/498,913, filed Apr. 28, 2023, which is hereby incorporated herein by reference.

### FIELD OF DISCLOSURE

This application relates to devices, instruments and methods for the treatment of various condition related to the vertebral disc space, and more specifically, to spinal fusion procedures and treatments. The devices cut, scrap or otherwise disrupt disc tissue and/or may serve as a fusion implant. The devices, optionally, include features such as lordosis locking features, expander plates, and robotic and/or motion control mechanisms.

### BACKGROUND

Spinal fusion is one of the primary treatment options for a variety of spinal conditions due to various degenerative, neoplastic, traumatic and infections processes. In general, achieving spinal fusion requires bony growth between two or more adjacent spinal vertebrae.

The need exists for a rapid, more thorough, and safer method to evacuate the spinal disc material in preparation for interbody fusion and efficient delivery of implants that reduce the number of components needed to prepare the disc space and deliver the implant.

### SUMMARY

In one aspect, a device for treating a intervertebral disc space includes a deployment device having a proximal end and a distal end. The device also includes a head releasably attached to the distal end of the deployment device. The head has a body having a top portion and a bottom portion. The head also includes a top rotatable blade associated with the top portion of the body and rotatable relative to the body and a bottom rotatable blade associated with the bottom portion of the body and rotatable relative to the body. The device includes a drive mechanism to rotate the top and bottom rotatable blades.

In another aspect, an intravertebral disc treatment device includes a head configured to be releasably attached to a delivery device. The head has a body having a top portion and a bottom portion. The head also has a top rotatable blade associated with the top portion of the body and rotatable relative to the body and a bottom rotatable blade associated with the bottom portion of the body and rotatable relative to the body. The head has recesses in the body configured to be releasably grasped by a deployment device.

In another aspect, a discectomy device or instrument includes a disc preparation device that, optionally, separates from the deployment device and remains in the disc space to serve as the fusion implant. In one embodiment, the device includes a head assembly that is configured to perform a discectomy and/or separates from the instrument and becomes the fusion implant.

In another aspect, the head becomes the permanent implant having aspects and features described herein.

In another aspect, the ability to provide a lordotic angle to restore the proper anatomical configuration of the spine, for example, at L4, L5 to S1 of the lower lumbar spine is disclosed. The lordotic angle is provided by the creation of a taller edge along one side of the rotating blade on one side of the head that establishes lordosis when the blades are rotated into the deployment position.

In a further aspect, the creation of taller edges along the rotating blades are on both the top and bottom blades of the head.

In yet another aspect, the creation of partial taller edges are along one of the top and bottom rotating blades of the head.

Another aspect relates to the addition of expanding plates on top of the rotating blades to either add extra thickness or provide also a lordotic angles in the manner previously disclosed. The expanding plate can be mounted and held in place with grooves and snap features to hold them in place.

A further aspect is that the expanding plates can be mounted onto the outer surfaces of the rotating blades with grooves, or snap features, or a combination of thereof.

Additional aspects and features described within are related to a locking mechanism to hold the rotating blades in a deployed state.

An aspect of the locking mechanism includes the insertion of a staple across corresponding holes in the top and bottom rotating blades. The insertion of the staple into the aligned holes upon proper deployment would prevent further rotation.

Another aspect of the locking mechanism relates to having a receptacle feature half on the top rotating blade and the other half on the bottom rotating blade and when rotated to the deployment position, the receptable features align to accept a blocking component such as a pin or screw.

Another aspect of the locking mechanism involves upon removal of the driving shaft for the rotation of the blades, the insertion of a small locking part with the distal end shape of the driving shaft but a proximal portion that is keyed to snap into a portion of the center body to lock it in place.

Another aspect of the locking mechanism involves a drive shaft extension member that can be active to lock the head in the deployed configuration. The drive shaft extension is associated with the drive shaft and is located in the head when the head is attached to the deployment device and the drive shaft is placed into the head. The drive shaft extension has a locking member or locking key component associated with the drive shaft extension. When the head is in the desired deployed configuration, the drive shaft member is activated to deploy the locking member or key. The head includes a corresponding receptacle that receives the locking member to lock the head in the deployed configuration. For instance, when the locking member is inserted, rotation of the heads is inhibited, locking the head in the deployment position. In one embodiment, the locking member is associated with the distal end of the draft shaft and when the drive shaft is activated to deploy the locking member is deployed. The locking member component may be deployed via a slide and/or trigger feature integrated into the driveshaft. In other embodiments, the locking member may be a releasable portion of the drive shaft or releasable extension of the drive shaft.

A further aspect involves the ability for the heads to be manually expanded to increase the overall height of the overall implant, wherein at least one of the heads are affixed to the extending member of the head via a ball and socket joint or similar spheroid joint. This ball and socket joint would allow the head to tilt and pivot in order to naturally conform to the patient's anatomical lordosis.

A further aspect involves multiple cutouts on the top and bottom sides of the head body that correspond with protruding cutting fins that extend from the top and bottom blades towards the head body. These cutouts allow passage of the cutting fins through the head body as the blades are rotated, wherein the sharp edges of the cutting fins provide multi-directional dissection and fragmentation of the disc material during rotation of the blades.

In addition, and further aspect relates to the addition of features to accommodate robotic and/or motion control of the discectomy instrument with its head via motion control system for navigation of the instrument and implant during surgery.

These and other aspects will be apparent from the following description.

### BRIEF DESCRIPTION OF DRAWINGS

FIG. 1 is a schematic top view of a head of the present disclosure implanted into a disc space.

FIG. 2 is a schematic side view of the spine with the head of FIG. 1 implanted into the disc space.

FIG. 3a is a perspective view of one embodiment of a discectomy instrument in accordance with the present disclosure, including a deployment device and detachable head.

FIG. 3b is a cross-sectional view of the deployment device of FIG. 3a.

FIG. 3c is a cross-sectional view of the proximal end of the deployment device of FIG. 3a.

FIG. 3d is a cross-sectional view of the deployment device of FIG. 3a.

FIG. 3e is an exploded view of the deployment device of FIG. 3a, showing the outer sleeve, the grabbing sleeve and the drive shaft.

FIG. 3f is a perspective view of a deployment device showing optional visual indicators for indicating the position of the rotating blades during use.

FIG. 4 is a perspective bottom view of the head of FIG. 3a with the shaver blades deployed or in a rotated configuration.

FIG. 5 is a perspective top view of the head of FIG. 3a with the shaver blades deployed or in a rotated configuration.

FIG. 6 is a perspective view of a discectomy instrument including a deployment device and a head.

FIG. 7 is a partial perspective view of the head of FIG. 6 shown without the main body to provide a clearer view of the grabbing arms and the drive mechanism.

FIG. 8 is a partial perspective view of the head of FIG. 6 shown without the main body and the grabbing arms to provide a clearer view of the drive mechanism.

FIG. 9 is a partial perspective view of the head of FIG. 6 shown without the top shaver blade to provide a clearer view of the drive mechanism.

FIG. 9a is a partial perspective view of the head of FIG. 6 shown without the body to provide a view of another embodiment of the drive mechanism.

FIG. 10 is a perspective view of the distal end of the instrument of FIG. 6 showing the head attached to the deployment device.

FIG. 11 is a perspective view of the distal end of the instrument of FIG. 6 shown without the top and bottom blades and the grabbing arms of the deployment device.

FIG. 12 is a perspective view of the distal end of the instrument of FIG. 6 shown without the grabbing arms and the drive shaft mechanism of the deployment device.

FIG. 13 is a side view of an embodiment of a head with an elevated edge to create lordosis on the top blade.

FIG. 14 is a side view of the head of FIG. 13 with the elevated edge to create lordosis on only one side.

FIG. 15 is a perspective view of the head of FIG. 13 with an elevated edge to create lordosis.

FIG. 16 is a side view of another embodiment of a head with a partial elevated edge to create lordosis on the top and bottom blades.

FIG. 17 is an end view of the head of FIG. 16 with a partial elevated edge to create lordosis on the top and bottom blades.

FIG. 18 is a perspective view of the head of FIG. 16 with a partial elevated edge to create lordosis on the top and bottom blades.

FIG. 19 is a schematic top view of the head of FIG. 13 with the elevated edge assembly and its instrument engaged into a disc space undeployed.

FIG. 20 is a schematic top view of the head of FIG. 13 with the elevated edge assembly and its instrument engaged into a disc space in a deployed position.

FIG. 21 is a schematic side view of the head of FIG. 13 with the elevated edge assembly and its instrument in a deployed position showing the created lordosis.

FIG. 22 is a schematic top view of the head of FIG. 16 with a partial elevated edge assembly with its instrument engaged into a disc space.

FIG. 23 is a schematic top view of the head of FIG. 16 with a partial elevated edge assembly with its instrument engaged into a disc space in a deployed position.

FIG. 24 is a schematic side view of the head of FIG. 16 with a partial elevated edge assembly with its instrument engaged into a disc space showing the created lordosis.

FIG. 25 is a perspective view of another embodiment of a head.

FIG. 26 is an exploded view of the head of FIG. 25.

FIG. 27 is an end view of the head of FIG. 25.

FIG. 27a is a cross-sectional view of the head shown in FIG. 25.

FIG. 28 is a top view perspective view of the head of FIG. 25.

FIG. 28a is a cross-sectional view of the head shown in FIG. 28 with the locking mechanism positioned within the head.

FIG. 29 is a perspective view of an head assembly with grooves on the top blade which accept an expansion plate.

FIG. 30 is an end view of the head assembly of FIG. 29 with top and bottom blades including grooved features or dove-tailed slots to accept expansion plates.

FIG. 31 is a top view of the head assembly of FIG. 25 with grooves on the top blade to accept an expansion plate including dimples to accept locking elements.

FIG. 32 is a perspective view of one embodiment of an expansion plate with dove-tailed grooves and locking elements.

FIG. 33 is a cross sectional view of the expansion plate of FIG. 28 that is of a flat/parallel and uniform shape with the locking elements.

FIG. 34 is a cross sectional view of another embodiment of an expansion plate that has a lordotic shape with locking elements.

FIG. 35 is a perspective view of one embodiment of a locking element.

FIG. 36 is a perspective view of another embodiment of a head assembly with retention holes for snap features on the top blade to accept an expansion plate.

FIG. 37 is a cross sectional view along line A-A, which illustrates the snap features on the top and bottom blades to accept an expansion plate.

FIG. 38 is a perspective view of one embodiment of an expansion plate showing the mating surface of the expansion plate having the snap features.

FIG. 39 is a side view of another embodiment of an expansion plate having a lordotic shape on one side and snap features on the other side.

FIG. 40 is a perspective view of one embodiment of snap features used for retention of the expansion plate on a shaver blade.

FIG. 41 is a representation of one embodiment of a locking staple device for preventing rotating movement of the shaver blades.

FIG. 42 is a cross sectional view of the head assembly with the locking staple and an inserting instrument.

FIG. 42a is a perspective view of the locking staple and inserting shaft.

FIG. 43 is a cross sectional view of the head assembly showing the locking mechanism having a receptacle feature in half of the top rotating blade and half of the bottom rotating blade.

FIG. 44 is a cross sectional view of the head assembly the position of the receptacle and the locking set screw to complete the locking.

FIG. 45 is the posterior end view of the head assembly where the receptacle is located.

FIG. 46 is a cross sectional view of the head assembly with locking shaft located in the receptacle preventing any further rotation.

FIG. 47 is a perspective view of the locking shaft.

FIG. 48 is an exploded perspective view of a head with another embodiment of a locking mechanism.

FIG. 49 is a perspective view of the discectomy instrument including the detachable head and adapters/interfaces to enable the device to be driven and controlled via robotic control system.

FIG. 50 is a representation of the tilting head attached to the extending member via a ball and socket joint.

FIG. 51 is a representation of the head body with cutouts that correspond to cutting fins protruding from the top and bottom blades.

FIG. 52 is a perspective view of a head and a bone graph delivery system.

FIG. 53 is a perspective view of a head and another embodiment of a bone graph delivery system.

FIG. 54 is a perspective view of a head and another embodiment of a bone graph delivery system.

### DETAILED DESCRIPTION

The devices, instruments, and methods disclosed herein may be utilized with the devices, instruments, and methods of those described in U.S. patent application Ser. No. 17/143,260, filed Jan. 7, 2021, published as US 20210212712, which is hereby incorporated by its entirety by reference herein.

The devices and methods disclosed herein generally relate to the deployment of a head of an intervertebral disc instrument into the disc space. The head may be a shaver and/or a spinal fusion implant. For example, the head may be used as a shaver to perform a discectomy and then implanted in the disc space to serve as spinal fusion implant. Thus, in some embodiments, the head is a detachable head that is a combination discectomy/implant device. In some embodiments, the devices and methods have the ability to create lordosis using an elevated edge or section on the top and/or bottom of the head.

In some embodiments, the lordotic angle can be accomplished by having a partially elevated edge or section on one of the top or the bottom of the head or a partially elevated edge or section on each of the top and bottom of the head. This may assist the treatment of deformity of the spinal column to restore proper lordotic anatomy, which may be in the lower lumbar spine.

In some applications a taller head having a greater height in the direction of the spine may be desired for insertion into the disc space. In such applications, expansion plates may be associated with one side or both sides of the head, with or without a lordotic angle, as set forth in more detail below.

In some embodiments the expansion plates are associated with the head by dove-tailed grooves. Optionally, the expansion plates may be locked or attached to the head with a locking feature, including but not limited to, ball-nose spring plungers or the like. In another alternative, the expansion plates may be attached by one or more snap-in plug features that retain the expansion plate in place. More details of the expansion plates will be described later.

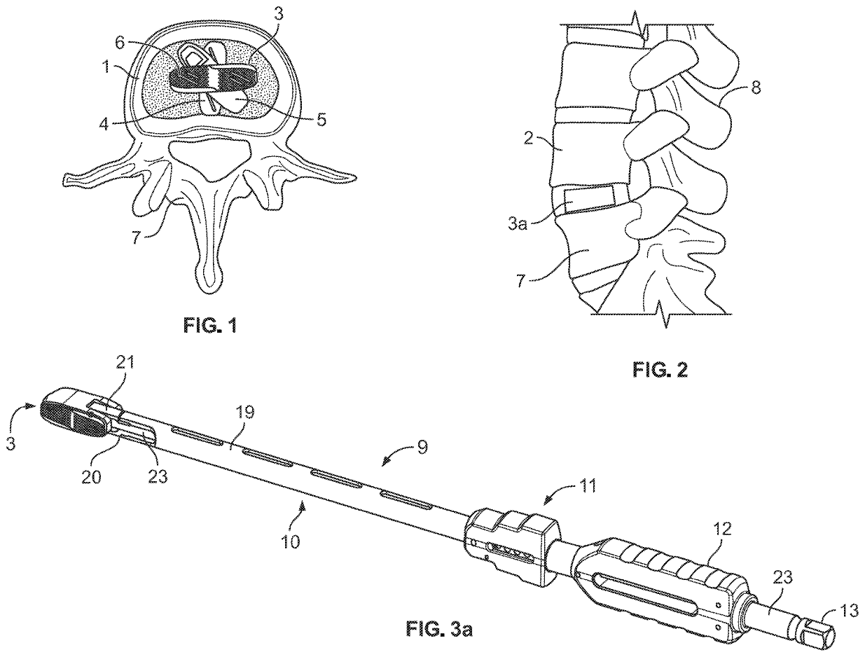

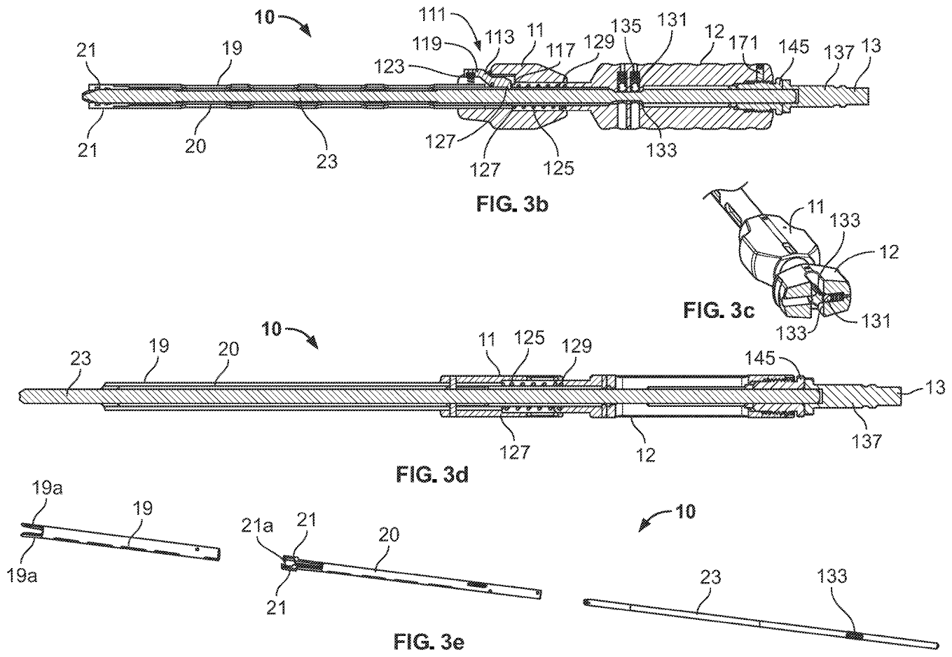

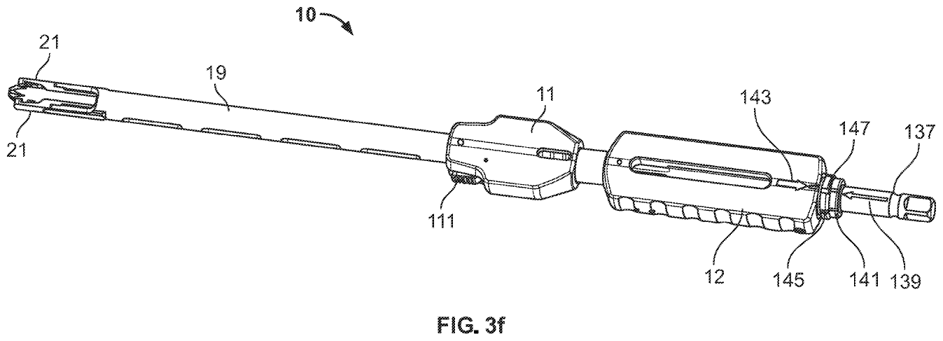

Turning to FIGS. 1 and 2, there is shown a schematic view of a disc space 1 having the head 3 or 3a of the intervertebral treatment instrument or device 9 (FIG. 3a) deployed as an implant for restoring disc height and to create a fusion bridge or structure between the upper vertebral body 2 and the lower vertebral body 7 of spine 8. It will be understood that the head 3 of the device 9 may function as a discectomy instrument, implant, or both. For example, head 3 may be a shaver head that cuts, tears, scrapes or otherwise disrupts tissue of the spine, such as intervertebral disc tissue and endplate tissue of vertebral bodies. The head 3 also may be detached or released from the deployment device 10 of the instrument 9 while head 3 is in the disc space 1, where the head 3 remains and serves as a fusion implant.

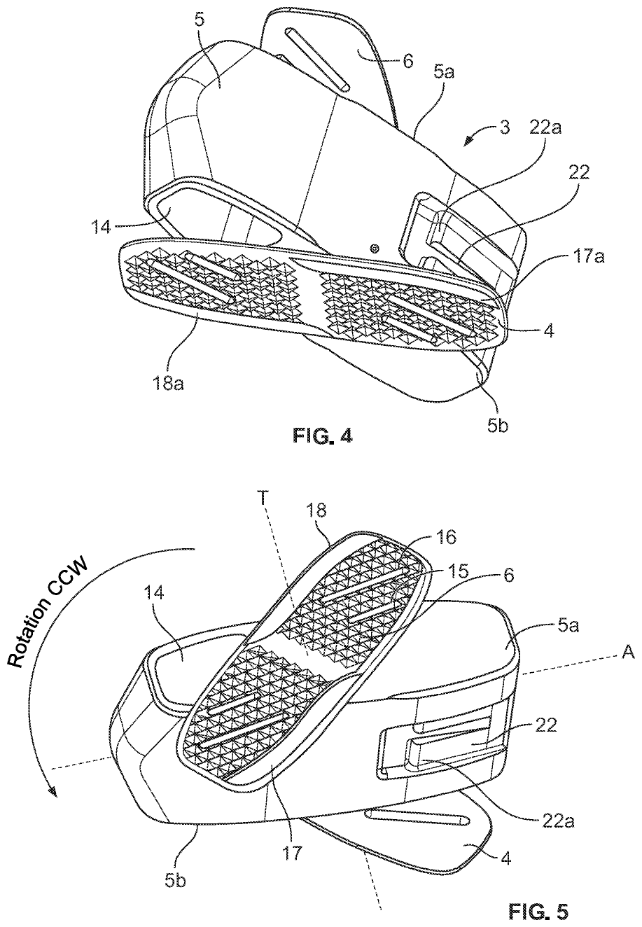

Referring to FIGS. 4 and 5, head 3 has a body 5 which a top portion 5a and a bottom portion 5b. Head 3 includes a top blade 6 associated with the top portion 5a of body 5 and a bottom blade 4 associated with the bottom portion 5b of body 5. Blades 4 and 6 rotate about a transverse axis T (FIG. 5) that is perpendicular to the longitudinal axis A (FIG. 5) of body 5. In the undeployed or initial configuration, the blades 4 and 6 are aligned with the body 5 such the longitudinal axes of the blades are parallel with the longitudinal axis A of body 5. Also in some embodiments, the proximal ends of the blades are aligned with the proximal end of the body and the distal ends of the blades are aligned with the distal end of the body, as shown in FIG. 3a.

In an implanted configuration, the top blade 6 and bottom blade 4, optionally, may be rotated relative to body 5 so as to be misaligned with or askew relative to body 5 (FIGS. 1, 4 and 5). For example, the longitudinal axes of the top blade 6 and bottom blade 4 are non-parallel with the longitudinal axis A of body 5. In some embodiments, the top blade 6 and bottom blade 4 are misaligned with body 5 and are aligned with each other. In some embodiments, the top blade 6 and bottom blade 4 are misaligned with or askew relative to body 5 and/or relative to each other. That is, in some embodiments, the longitudinal axis of the top blade and the longitudinal axis of the bottom blade are non-parallel. Optionally, the longitudinal axis of the top blade, the longitudinal axis of the bottom blade and the longitudinal axis A of the body are all non-parallel relative to one another. In some embodiments, when the top and bottom blades 6, 4 are misaligned, the proximal and distal ends of the blades are not aligned with each other. In some instances, the blades form an X-like configuration. This misalignment of the blades and body creates a large surface area which aids in preventing subsidence.

Referring back to FIG. 3a, the fusion instrument with its head 3 is generally designated by reference numeral 9. Fusion instrument 9 may be a discectomy instrument and/or an implant delivery instrument. For example, instrument 9 may be used as a discectomy instrument to evacuate tissue from the disc space.

Instrument 9 could be used to delivery fusion implant (head 3) into the disc space. Alternatively, instrument 9 could be used as both a discectomy instrument and an implant delivery instrument.

The head 3 is located at the distal end of the instrument 9 and is operatively connected to deployment device 10 with a controlling section 11 and the handle 12 of. In addition, deployment device 10 (and thus instrument 9) includes an interface 13 at the proximal end of a drive shaft 23 for connection to a handle (not shown) or robotic system for manipulating and controlling instrument 9. For example, the interface 13 may include a square connector to attach a T-handle or the robotic system. Referring to FIGS. 3a-3e, deployment device 10 includes a shaft assembly with an outer sleeve 19, a grabbing sleeve 20 and a drive shaft 23. The outer sleeve 19, grabbing sleeve 20, and drive shaft 23 are concentrically positioned relative to one another.

Referring to FIG. 4, the head (shaver head/implant) 3 is shown in the implanted configuration with the top blade 6 and the bottom blade 4 in an open, misaligned, or rotated position. The body 5 can have various heights so as to be suitable to treat a large variety of disc spaces. For example, the surgeon may select a body 5/head 3 having a particular height depending on the anatomy of the patient and the desired outcome of the procedure. Optionally, body 5 has a cavity 14 to accommodate bone graft material to enhance fusion. Turning to FIG. 5, top blade 6 may include tissue disruption members or features that cut, scrape, or otherwise disruption tissue within and/or adjacent to the disc space. The disruption members may be scraping members, sharp edges or points, etc. In the illustrated embodiment, the top or outer surface of top blade 6, optionally, may include scraping members for scraping or preparing the endplates of the vertebra adjacent the disc space for fusion. For example, the top surface of top blade 6 may include projections 15 and recesses 16. In some embodiments, the top surface of top blade 6 has a rasp like pattern or surface for scraping of the endplate. When recesses 16 are included, such recesses may allow loose material to accumulate and be removed upon retrieval of the head 3.

In FIG. 5, top blade 6 is shown rotating counter-clockwise, as shown by the arrow. Top blade 6, optionally, includes a sharp edge or cutting edge 17 extending at least partially along one longitudinal side of the blade 6 as well as another sharp edge or cutting edge 18 extending at least partially along the longitudinal opposite side of the top blade 6. The sharp edges 17 and 18 are on the leading portions of the top blade 6 during counter-clockwise rotation thereof. If the blade 6 were to rotate clockwise, the sharp edges 17 and 18 would be located on the leading portions of the sides of the blade. The sharp edges 17 and 18 cut the disc material apart from the endplate. Optionally, sharp edges 17a and 18a of the same or similar configuration are also found on the bottom blade 4, as shown in FIG. 4. Optionally, the bottom or outer surface of bottom blade 4 may also include scraping members. Additionally, the bottom blade 4 and top blade 6 may also include sharp edges along the longitudinal length of both sides of the blade. In such embodiments, the blades 4 and 6 could be alternately rotated in both a clockwise and counterclockwise direction to cut or otherwise disruption disc tissue. In some embodiments, several rotations of the blades may need to be carried out by the surgeon to completely clean-out the endplate. Optionally, the rotations may be in alternating clockwise and counterclockwise directions.

FIGS. 3a-3e and 6-9 illustrate embodiments of deployment device 10. Turning to FIGS. 3b-3e, there is shown an embodiment of deployment device 10 which includes an outer sleeve 19, a grasping sleeve 20, and a drive shaft 23. The outer sleeve 19, grasping sleeve 20 and drive shaft 23 may be concentric. For example, in the illustrated embodiment, the grasping sleeve 20 is positioned with the outer sleeve 19, and the drive shaft 23 is positioned within the grasping sleeve 20.

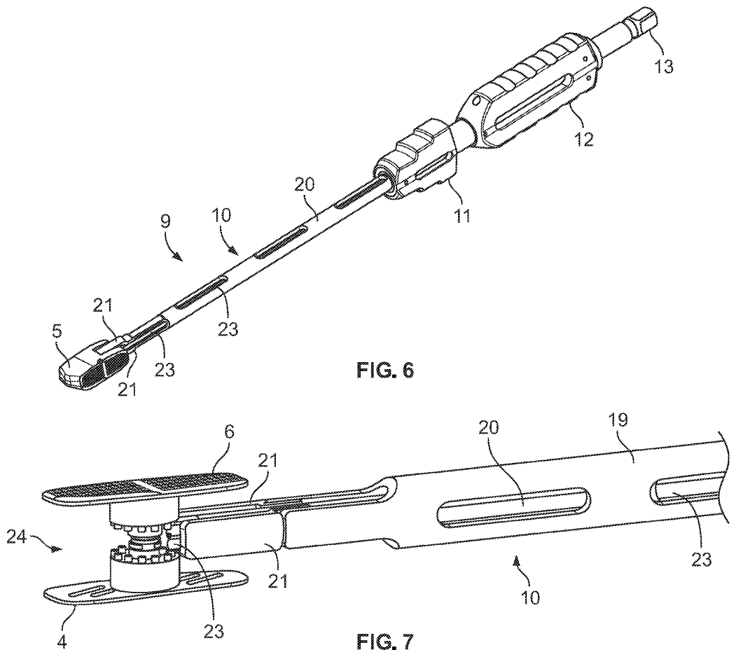

Turning to FIG. 6, the outer sleeve 19 of deployment device 10 is removed to show the other components at the distal end of instrument 9 and deployment device 10. As shown in FIGS. 6, 3b and 3e, deployment device 10 includes grabbing sleeve 20 having two distal prongs 21 for releasably engaging and holding head 3 (FIGS. 3a and 6). Grabbing sleeve 20 provides a releasable attachment mechanism for holding and releasing the head 3. Prongs 21 engage recesses 22 (FIG. 5) of head 3. Prongs 21 engage recesses 22 to secure the body 5 in place while deploying head 3 into the disc space and performing the discectomy. Optionally, instrument 9 may be deployed through an access sleeve (not shown) that may include trocars for creating access to the disc space. In other embodiments, the instrument 9 may be placed into the disc space in an open procedure without the use of an access sleeve.

The recesses 22 of body 5 may include one or more latches 22a (FIGS. 4 and 5) and the prongs 21 may include hooks 21a (FIG. 3e) that grasp or hold the latches 22a. Prongs 21 have an initial grasping configuration and a release configuration. In the grasping configuration, prongs 21 grasp and hold head 3. In the release configuration, prongs 21 spread apart from one another to release head 3. In some embodiments, prongs 21 are biased outward toward the release configuration. The biasing may be by a spring or by the material and structure of the prongs. For example, the prongs 21 may be made from an elastic material wherein the prongs may be forced toward each and when the force is removed, the prongs 21 move away from each other due to the elastic material of the prongs.

The grabbing sleeve 20 is positioned in the outer sleeve 19 so that the outer sleeve 19 at least partially covers prongs 21 and forces them toward each in order to grasp and hold head 3. Optionally, the outer sleeve 19 may include projections 19a (FIG. 3a) at the distal end of the outer sleeve, wherein the projections 19a are aligned with and contact prongs 21 to hold the prongs in the grasping configuration. That is, outer sleeve 19 covers at least a portion of the prongs 21 pushing the prongs together and into recesses 22.

The outer sleeve 19 may be slid axially toward the proximal end of the instrument 9. In the illustrated embodiment, the controlling section 11 is operatively connected to the outer sleeve 19, and sliding of outer sleeve 19 is accomplished by moving the controlling section 11. When the outer sleeve 19 is slid axially and proximally, it uncovers the prongs 21 a sufficient amount, removing the force from the prongs 21. With the force removed, grabbing prongs 21 spread apart, thereby removing the prongs from the recesses and releasing the body head 3.

Referring to FIG. 3b, controlling section 11 may have a releasable locking mechanism 111. Locking mechanism 111 includes a lever 113 that pivots about a pivot point 115. Lever 113 includes a detent 117 at one end of lever 113 and a depressible portion 119 at the other end. The detent 117 engages a recess 121 in the gasping sleeve 20 to hold the outer sleeve 19 in the grasping configuration, wherein the outer sleeve forces prongs 21 of the grasping sleeve 20 into the grasping configuration. A biasing member, such as spring 123, biases the lever 113 so that the detent 117 engages the recess 121. The controlling section 11 is biased distally and to the grasping configuration by a spring 125 that sits between a shoulder 127 of the controlling section 11 and a shoulder 129 of the handle 12. To move the grasping prongs 21 to the releasing configuration. The user pushes down the depressible portion 119 of lever 113. Pushing down depressible portion 119 rotates lever 113 and removes detent 117 from recess 121. The user then pulls controlling section 11 proximally, thereby moving outer sleeve 19 proximally and uncovering prongs 21 of grasping sleeve 20. With the outer sleeve 19 moved, the prongs 21 spread apart, thereby releasing head 3.

Referring to FIGS. 6-10, the deployment device 10 includes rotating drive shaft 23 that spans from the head body 5 to the T-handle interface 13. In use a surgeon holds the handle 12 while rotating the T-Handle (not shown), which in turn will rotate the drive shaft 23 within outer sleeve 19 and grasping sleeve 20. Rotation of Drive shaft 23 rotates the top and bottom blades 6, 4 of head 3 at the distal tip of the instrument 9. The operative connection between the rotating drive shaft 23 and the top and bottom blades 6, 4 may be the same or similar to that disclosed in U.S. patent application Ser. No. 17/143,260, filed Jan. 7, 2021, published as US20210212712, which incorporated by reference herein. For example, the rotating drive shaft 23 may be connected to head 3 by the same gear configuration and arrangement described in the above-mentioned reference. The rotating drive shaft 23 may be connected to the head 3 by any other suitable arrangement, such as that described below.

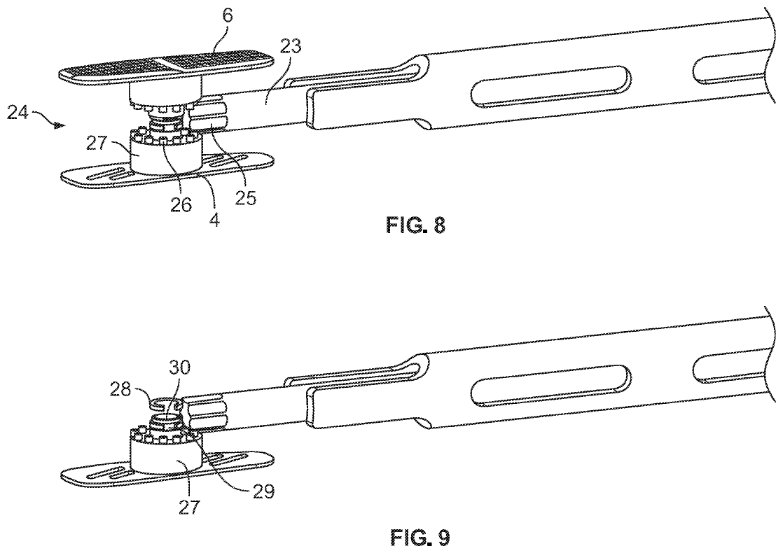

FIG. 7 shows the distal end portion of the instrument 9 with the body 5 of head 3 removed to expose how the top and bottom blades 6, 4 are activated and details of the drive mechanism 24. Referring to FIG. 8, the drive mechanism 24 includes a circumferential gear 25 at the distal end of the rotating drive shaft 23. Circumferential gear 25 engages posts 26 that are spaced on top of the shoulder 27 of the bottom blade 4. It is understood that circumferential gear 25 and the posts 26 are acting like a bevel gear set. In the illustrated embodiment, the top blade 6 has the same exact pattern of posts 26 so that rotating drive shaft 23, will cause the top and bottom blades 6, 4 will rotate in opposite directions and at the same rate of rotation due to the acting of the bevel gear set. Looking at the drawings, the top blade 6 will rotate counter-clockwise and the bottom blade 4 will rotate clockwise. In other embodiments, the posts 26 associated with the top and bottom blades 6, 4 may vary relative to each other such that the blades rotate at different rates.

Turning back to FIGS. 3b, 3c and 3e, the deployment device 10, optionally, includes elements that provide for incremental rotation of drive shaft 23, which results in incremental rotation of blades 4, 6. In the illustrated embodiment, handle 12 includes detents 131 that engage recesses 133 in drive shaft 23. The recesses 133 are spaced circumferentially round the shaft 23. The number and spacing of the recesses 133 may be varied depending on the desired incremental rotation of the shaft 23. The detents 131 are biased into recesses 133 by a biasing member, such as spring 135. To rotate drive shaft 23, sufficient rotational force is applied to overcome the force of the detents 131 engaged with the recesses 133. When such rotational force is applied, the drive shaft 23 pushes detents 131 upward allowing an incremental rotation of the drive shaft. As the drive shaft 23 rotates and the detent 131 engages the next recesses 133, there is at tactile or noise indication that the drive shaft 23 has been rotated one increment. The increments assist the user in knowing what position the blades 4 and 6 are in.

Turning to FIG. 3f, optionally, the deployment device 10 may also have a visual indicator indicating the position of the head's blades 4, 6. In the illustrated embodiment, a hub 137 is attached to the proximal end of shaft 23 (FIGS. 3b, 3c and 3e). Tuning to FIG. 3f, the hub 137 rotates with shaft 23 and includes visual markings 139 and/or 141. Marking 139 is shown as an arrow and marking 141 are tick marks. In the illustrated embodiment, when arrow 139 is aligned with an arrow or other visual marking 143 on handle 12, the head is in the undeployed configuration and may be inserted and/or removed from the disc space. The handle includes a collar 145, in which the drive shaft 23 rotates. The collar 145, handle 12 or any other portion of the deployment device may also have a visual mark 147 to which tick marks 141 can be compared. The comparison of tick marks 141 to visual mark 147 provides a visual indication of the position of the head's blades 4 and 6.

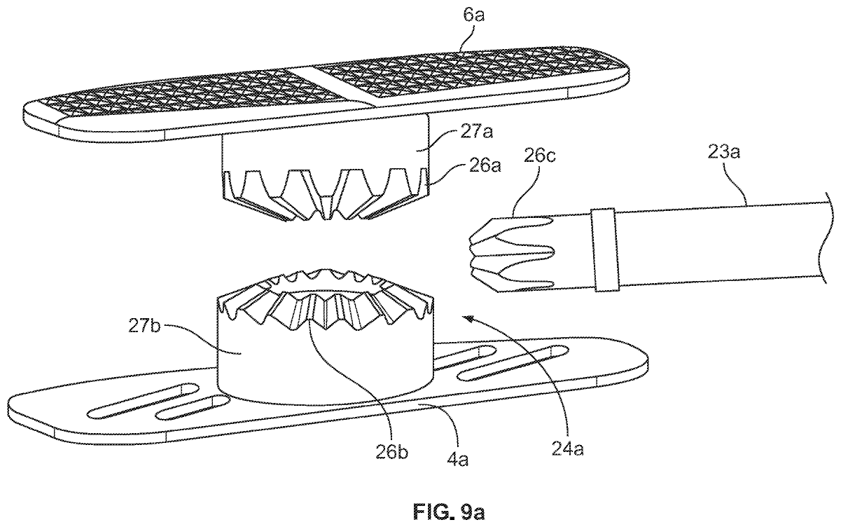

Turning back to FIG. 9, a pair of snap rings 28 within a groove receptacle 29 are located on shaft 30 atop of the shoulder 27 on one side. A corresponding groove 31, shown in FIG. 11, is located on the body 5. There is one snap ring 28 associated with the top blade 6 and one associated with the bottom blade 4.

FIG. 9a illustrates another exemplary drive mechanism 24a, which is a miter gear assembly. Top blade 6a has gear teeth 26a or a toothed-bearing face on shaft 27a, and bottom blade 4a has gear teeth 26b or a toothed-bearing face of shaft 27b. Another set of teeth 26c or toothed-bearing face is located at the distal end of drive shaft 23a. The teeth spacing of the teeth may vary depending on the rate of rotation desired for a particular application. Similar to drive shaft 23, rotation of drive shaft 23a rotates blades 4a, 6a. The miter gear provides sufficient force and torque to disrupt and remove disc tissue.

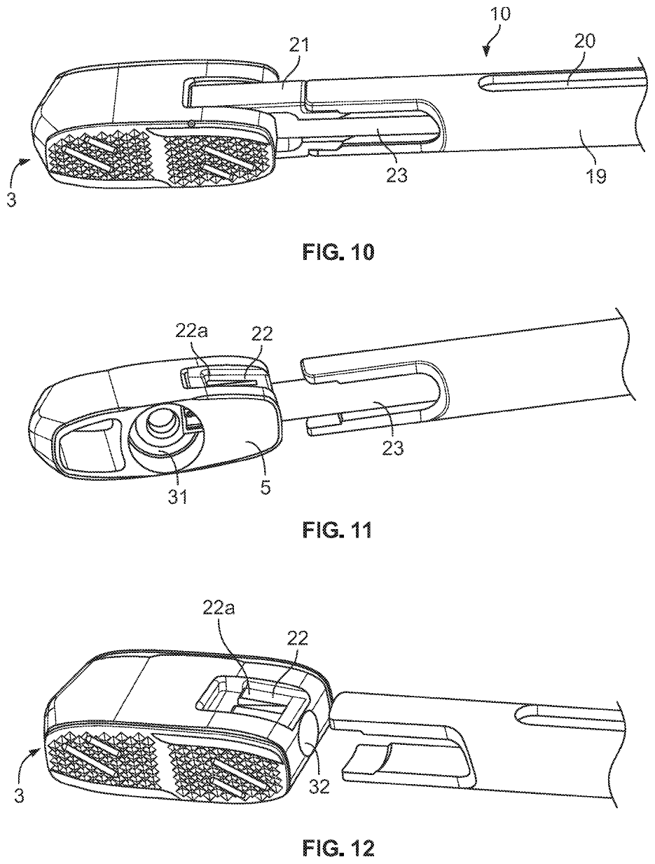

FIG. 10 shows head 3 with the deployment device 10 having outer sleeve 19, distal prongs 21 of the grabbing sleeve 20 and rotating drive shaft 23.

Referring to FIG. 11, the grabbing sleeve 20 has been removed from this figure so as to show the engaging recess 22 and latches 22a of head 3. The recess 22 is where the distal prong 21 would be located for holding the head 3 in place. FIG. 12 shows another view of the recess 22 and the hole 32 that houses or is for insertion of the distal end of the rotating drive shaft 23 that was shown in FIG. 10 and FIG. 11.

Turning to FIG. 13, this figure shows a side view of another embodiment of a head 36 where the top edge 33 of the top rotating blade 6 is raised to create a lordotic angle to be used in certain situations wherein spinal lordosis is desired or is one of the goals of the surgery to restore the proper anatomy.

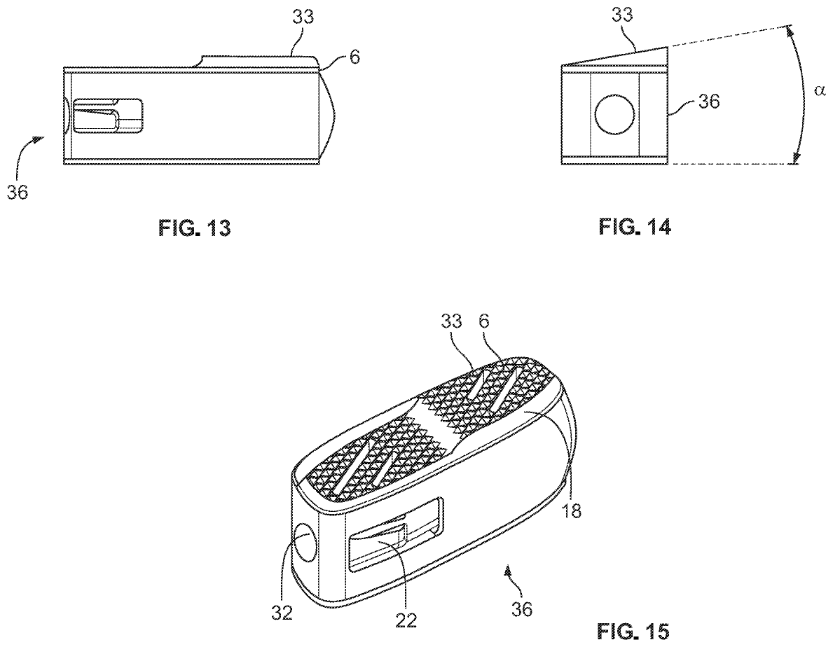

FIG. 14 shows the end view of head 36 with the raised edge 33 that creates the lordotic angle a, which in some embodiments could be from 1 degrees to 20 degrees as measured from a plane that is parallel with the bottom surface of the body 5. In some embodiments, the angle a may be between about 6-8 degrees. FIG. 15 shows the perspective view of head 36 and the angled top rotating blade 6 with the raised edge 33 and also the sharp edge 18 for disrupting disc material as previously described.

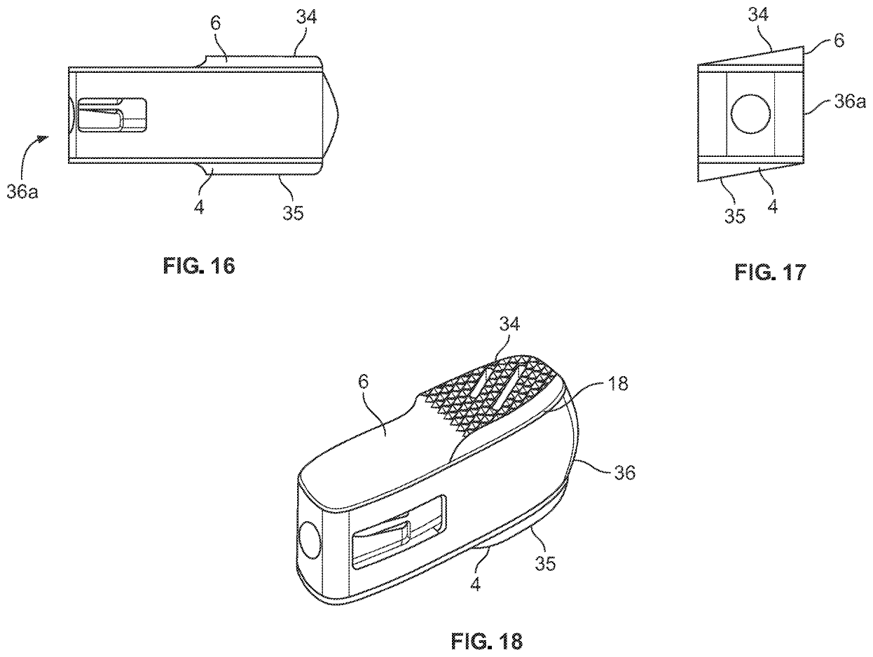

FIG. 16 shows another embodiment of head 36a for creating a lordotic device with a partial raised edge 34 on the top side and another partial raise edge 35 on the bottom side. FIG. 17 shows an end view of the head 36a and when the blades 4, 6 are aligned with body 5 in the undeployed configuration, raised edges 34, 35 are in opposite directions or on opposite sides relative to the longitudinal midline of body 5. When in the deployed configuration, the blades are misaligned and the raised edges 34, 35 will be in the same direction as shown in FIG. 23 and FIG. 24 and will be described later.

FIG. 18 shows a perspective view of the head 36a and the partial angled top rotating blade 6 with the raised edge 34 and also the sharp edge 18 for disrupting disc material as previously described. It can also be observed the partial raised edge 35 of the bottom rotating blade 4.

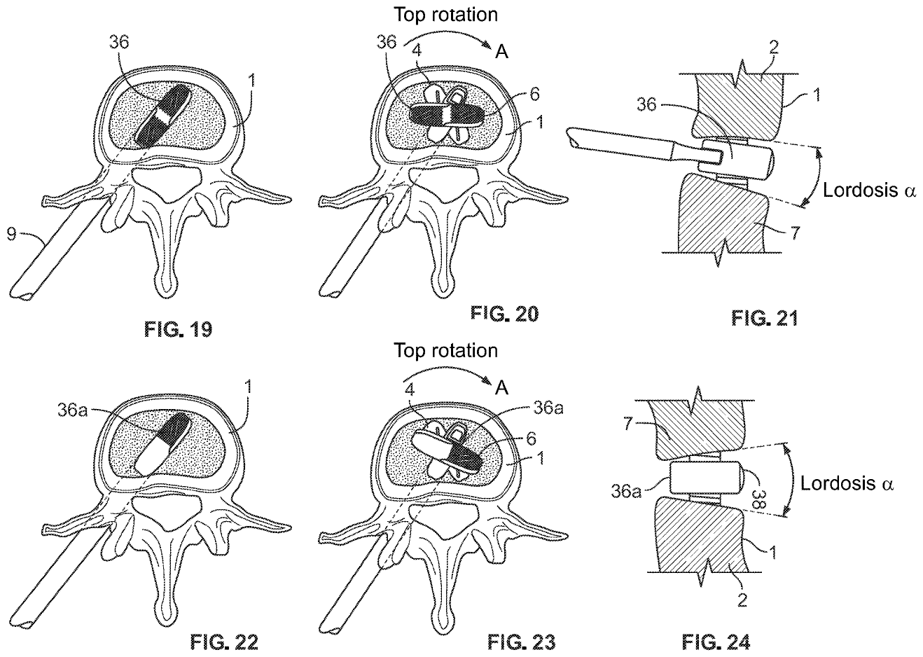

FIGS. 19-21, show schematic steps of the deployment for the head (serving as a lordotic device) of FIG. 15. FIG. 19 shows the insertion of the discectomy instrument 9 with its head 36 into a disc space 1 in the undeployed state. The discectomy instrument 9 may be used to deploy any of the heads disclosed herein. The discectomy instrument may be used in minimally invasive procedures that using access trocars and tubes. In such procedures, the head may be inserted through the access tube and into the disc space. The discectomy instrument 9 may also be used in open surgical procedures. Furthermore, the discectomy instrument may be used to insert the head in an orientation wherein the transverse axis T is perpendicular to the longitudinal axis of the spine. When inserted in this manner, the sides of the body of the head contact the endplates of the vertebrae above and below the disc space. This may assist in forcing the vertebrae apart and restoring the height of the disc space. After the head has been placed in the disc space in this orientation, the instrument may be rotated 90 degrees to rotate the head 90 degrees, thereby facing the top and blades to the endplates. Additionally, in this orientation, the transverse axis T is parallel with the longitudinal axis of the spine. In some embodiments, the top and bottom blades contact the endplates.

Furthermore, any of the heads disclosed herein may be placed in the disc space in the undeployed or initial configuration. In the disc space, the blades may be rotated to disrupt disc tissue (cut, scrape, tear, or otherwise disrupt). The head may be placed back in the undeployed configuration, wherein the disrupted disc tissue is caught, trapped, snagged, or otherwise grasped by the head. For example, the tissue may be caught between the blades and the body or in the rasp-like surfaces of the blades. In the undeployed configuration, the head may be removed from the disc, thereby removing the disrupted tissue caught in by the head. The surgeon may then clean the head and place it back into the disc space to disrupt more tissue or implant the head. Alternatively, after the head is moved from the disc space, the surgeon may release the head and replaced with a new head. The new head may be used to disrupt more tissue and be similarly removed, or the new head may be implanted in the disc space. The new head, and any other subsequent heads used, may be of the same configuration of the first or subsequent heads, or the new head may be of a different configuration.

For instance, a head(s) having a first configuration may be used to disrupt and remove tissue, and a head of the different configuration may be used as the implant. In one example, a first head that does not have lordotic elements may be employed to disrupt and remove tissue. After the first head has been removed, it may be replaced with a head with lordotic features that is implanted in the disc space. It will be understood that in any of the procedures, other tissue disruption and tissue removal devices may be used to disrupt and remove tissue. For example, other cutters, scrapers and tissue graspers may be used to remove tissue for preparing the disc space.

Referring to FIG. 20, the lordotic head 36 has been deployed with the top and the bottom rotating blades 6, 4 activated with the top blade 6 rotating in the rotation direction A and the bottom blade 4 rotating in the opposite direction. The blades rotate about the body of head 36 to disrupt and remove tissue. The blades are then stopped or positioned in the deployed configuration.

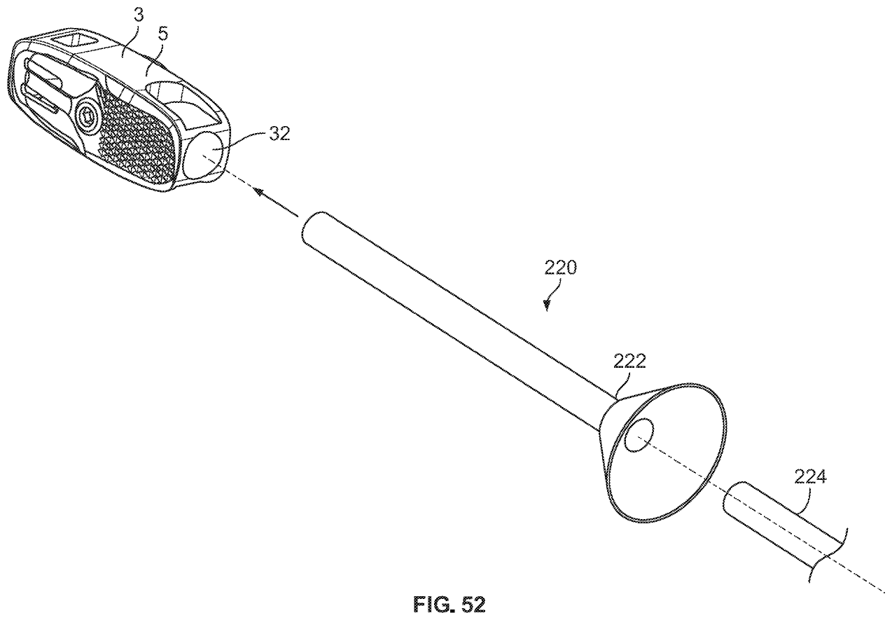

The lordotic angle a is shown in FIG. 21 which is a side view of the disc space 1 with the upper vertebral body 2 and the lower vertebral body 7 and the deployed head 36 showing lordosis is achieved. The head 36 is detached from the instrument 9 and remains in place, serving as a fusion implant. After the implant has been placed, the blades of the head may be locked and bone graft may be deployed into the implant as described below relative to FIGS. 52-54.

Turning to FIGS. 22-25, lordotic head 36a with the partial raised edges is delivered into the disc space 1 in the undeployed state. And similarly, as shown in FIG. 20, it can be seen in FIG. 23 that the lordotic head 36a has been deployed with the top blade 6 rotating in the rotation direction A and the bottom blade rotating in the opposite direction.

The lordotic angle a is shown in FIG. 24 which is a side view of the disc space 1 with the upper vertebral body 2 and the lower vertebral body 7 and the deployed shaved head 36a demonstrating how the creation of the lordosis is achieved and how the two partial raised edges align on the same side, meaning the anterior portion 38 of the disc space 1 to create the desired lordotic anatomical goal. The head 36a is detached from the instrument 9 and remains in place, serving as a fusion implant. After the implant has been placed, the blades of the head may be locked and bone graft may be deployed into the implant as described below relative to FIGS. 52-54.

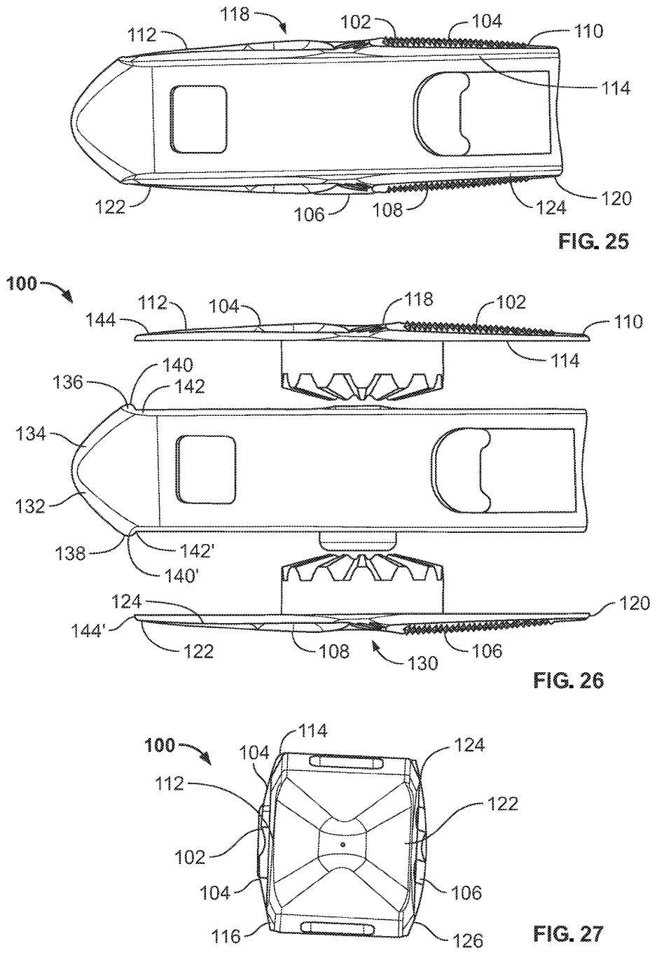

FIGS. 25-28a illustrate another embodiment of a head 100, which includes top blade 102 having a domed top surface 104 and/or a bottom blade 106 having a domed bottom surface 108. The domed top surface 104 has proximal end portion 110, a distal end portion 112 and opposed sides portions 114 and 116. The domed surface 104 is ramped or angled upward from the proximal end portion 110, distal end portion 112 and opposed side portions 114,116 to an apex 118. Surface 104 may have a convex shape. Similarly, the domed bottom surface 108 has proximal end portion 120, a distal end portion 122 and opposed sides portions 124 and 126. The domed surface 108 is ramped or angled upward from the proximal end portion 120, distal end portion 122, and opposed side portions 124, 126 to an apex 130. Surface 108 may have a convex shape.

Head 100 may have any of the features that were described above with respect to heads 3, 3a, 36 and 36a. For example, the blades 102 and 106 may have rasp-like features, disruption features, sharp edges, etc. Furthermore, the blades may be rotated by any of the drive mechanisms disclosed herein or any other suitable drive mechanisms.

The domed blades 102 and 106 may provide improved compliance with the anatomical profile of the disc surface for fusion procedures. For example, when the endplate surface is concave shaped, the domed or convex shape of the blades 102 and 106 may conform to the endplate surface, resulting in a more stable implant.

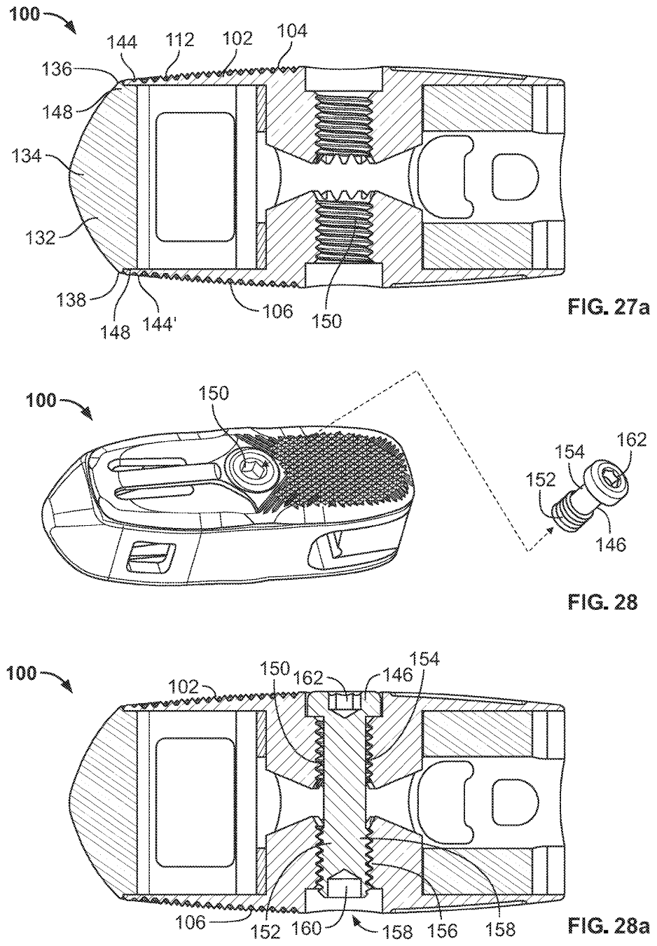

Referring to FIGS. 26 and 27a, the body 132 of the distal end 134 of head 100 may include top and bottom protection lips 136 and 138. The lips 136 and 138 include a shoulder 140, 140′ and a recess 142, 142′. The distal end portion 144 of the top blade 102 is located in recess 142 and below shoulder 140.

Likewise, the distal end portion 144′ of bottom blade 106 is located in recess 142′ and below shoulder 140. The lips 136 and 138 provide a smooth surface and protection against any potential material intrusion in between the rotating blades on the top and bottom side. As shown in FIGS. 27 and 27a, the lips provide a seamless bullet nose of the head, which shields the small gap 148 that is between the body 132 and the top and bottom blades 102 and 106. Any of the heads disclosed herein may, optionally, include the protective lips.

Turning to FIGS. 28 and 28a, any of the heads disclosed herein may include an axial insert 146 that engages a bore 150 of the head to hold securely the two rotating blades 102 and 106 onto the body and prevent looseness and wobbliness of the blades. Bore 150 may go through the blades 102 and 106 and the body of head 100. In the illustrated embodiment, the axial insert 146 includes a threaded portion 152 and unthreaded or smooth portion 154. Referring to FIG. 28a, the axial insert 146 is inserted into bore 150. The threaded portion 152 of axial insert 146 engages threads 156 of blade 106 and the smooth portion 154 resides in the portion of bore 150 defined by blade 102. The bottom portion 158 of axial insert 146 is permanently attached to blade 106. For example, the bottom portion 158 is swedged, welded, adhered with adhesive, etc. to blade 106. When swedging is employed, a swedging tool may be inserted into recess 160 in the bottom portion 158 of insert 146. The insert may also have a recess 162 for engaging a tool to screw the insert into the bore 150. When the blade 102 and 106 rotate relative to body, the axial insert 146 rotates with blade 106 and blade 102 rotates about the unthreaded portion 154 of the axial insert.

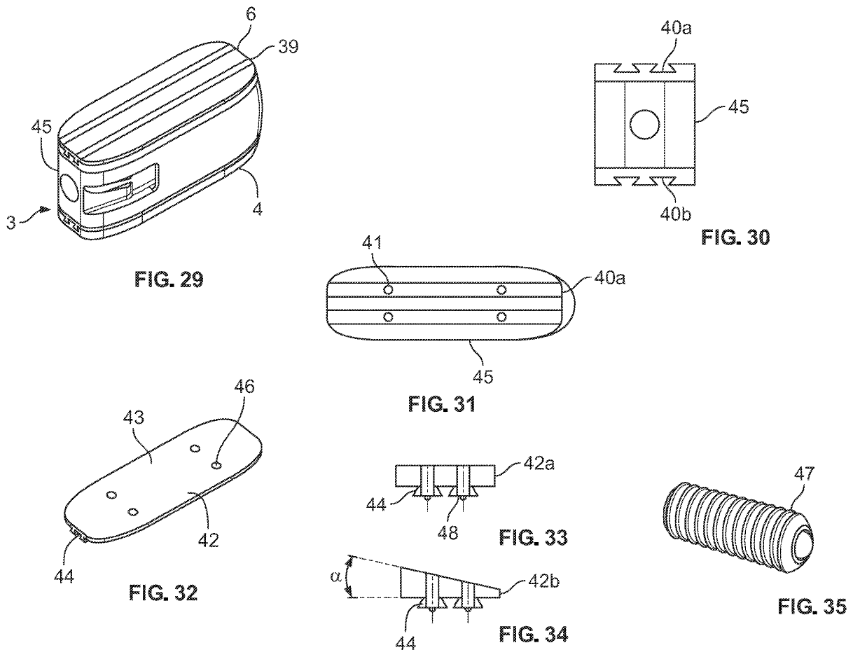

FIG. 29 illustrates an optional feature that could be used with any of the heads disclosed herein. Head 45 includes top blade 6 and bottom blade 4. One or both of the top blade 6 and the bottom blade 4 may have an expanding plate 42 associated therewith. In some embodiments, the top and/or bottom blades 6, 4 include one or more dove-tailed grooves 39. In the illustrated embodiment, top and bottom blades 6, 4 include two dove-tailed grooves 39. In other embodiment, only the top rotating blade has dove-tailed grooves or vice versa, only the bottom rotating blade has dove-tailed grooves. FIG. 30 is an end view that illustrates one optional placement of the dove-tailed grooves (40a at the top and 40b at the bottom) in the top and bottom rotating blades 6, 4 of the head 45.

FIG. 31 shows a top view of the head 45 in addition to the two dove-tailed grooves 40a, and the location of dimples or holes 41. The dimples 41 may be conical recesses in the bottom of each dove-tailed groove for the purpose of accepting a locking member, such as a ball-nose spring plunger, which securely attaches expanding plate 42 to the blade. In the illustrated embodiment, there are four dimples or holes 41.

The top portion 43 of the expanding plate 42 could have the same type of surface as previously described in FIG. 5 or any other alternative patterns that could benefit the purpose of the additional expander plate. A smooth surface or a rough surface could also be the finishes of the top of the expanding plate. Referring to FIGS. 32-34, at the bottom surface of the expanding plate 42 are located the dove-tails 44 that mate with the dove-tailed features 39 of the blades 4, 6. Dove-tails 44 have a shape configured to engage with the dove-tails 39 described in FIG. 29-31 . Referring to FIG. 32, there are four corresponding tapped through holes 46 to match the location of the recess dimples 41 previously described in FIG. 31. The tapped through holes may accept locking projections, such as ball-nose spring plunger 47, shown in FIG. 35 for instance.

The expanding plates 42 could have different configurations and different thicknesses to fit the desired application. In FIG. 33, an illustration of a parallel expanding plate 42 a is shown with a thickness of about 2-3 mm. In some embodiments, the thickness could range from 1 to 15 mm depending on the desired application. For example, depending on the height of the disc space. The mounted ball-nose spring plungers 47, 48 are also illustrated in FIGS. 33-35. In addition, another example of an expanding plate with a lordotic design 42b is shown in FIG. 34 and it is understood that the thickness could varies as suggested previously as well as the lordotic angle a could also varies as previously described in FIG. 14.

To mount the expanding plate onto the head 45, expanding plate 42, or 42a, and/or 42b is slid onto the corresponding mating dove-tailed features 39 until the ball-nose spring plungers 48 snap in place and retain the expanding plain in the proper position.

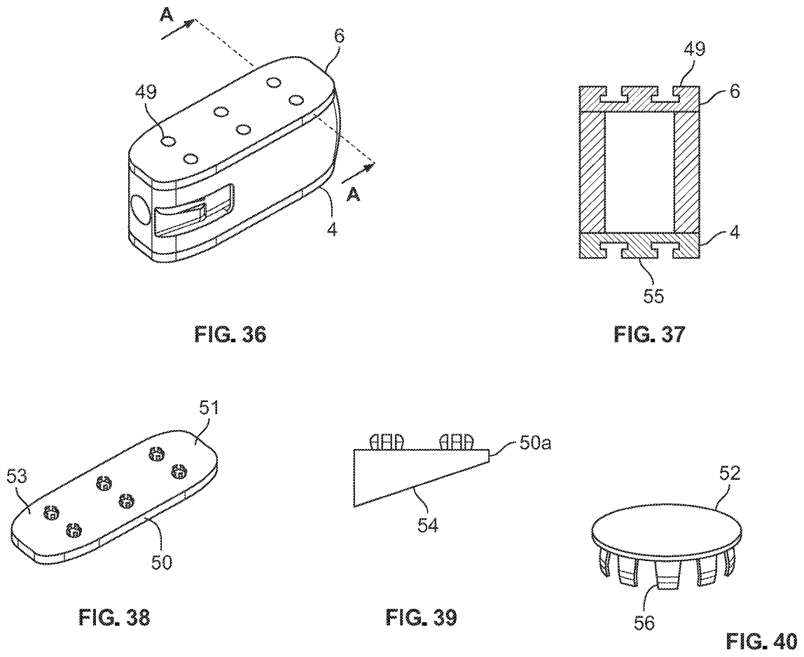

In some embodiments, expanding plates can be mounted onto the top of the rotating blades 6, 4 via snap features as shown in FIGS. 36-40. The rotating blades 6, 4 would have a number of recessed holes 49. In the illustrated embodiment, six are shown but any other number could be employed. The recess holes 49 are better shown in FIG. 37 which is a cross-section view at plan A-A. In the illustrated embodiment, recess holes 49 are located on the top rotating blade 6 as well as on the bottom rotating blade 4 but again, it is understood that only one rotating blade could accommodate an expanding plate either on the top side but also only one on the bottom side.

FIG. 38 shows an expanding plate 50 with the mating surface 53 that engages with the corresponding mating surface 55 of the bottom blade 4 shown in FIG. 37. Snap features 51 may be made of projections 56 shown in FIG. 40, which may be finger-like projections. For example, each snap feature may include at least two projections, and in some embodiments, include a plurality of projections. FIG. 40 shows one embodiment of a snap-in plug 52. Another snap-in configuration could also be used.

FIG. 39 shows an end view of the expanding plate 50a with snap-in features and a lordotic configuration. However, other configurations of the expanding plate can also be used as previously described. The mounting of such expanding plates includes positioning the snap-in features in line with the corresponding recess holes 49 and pressing the expanding place and blade together.

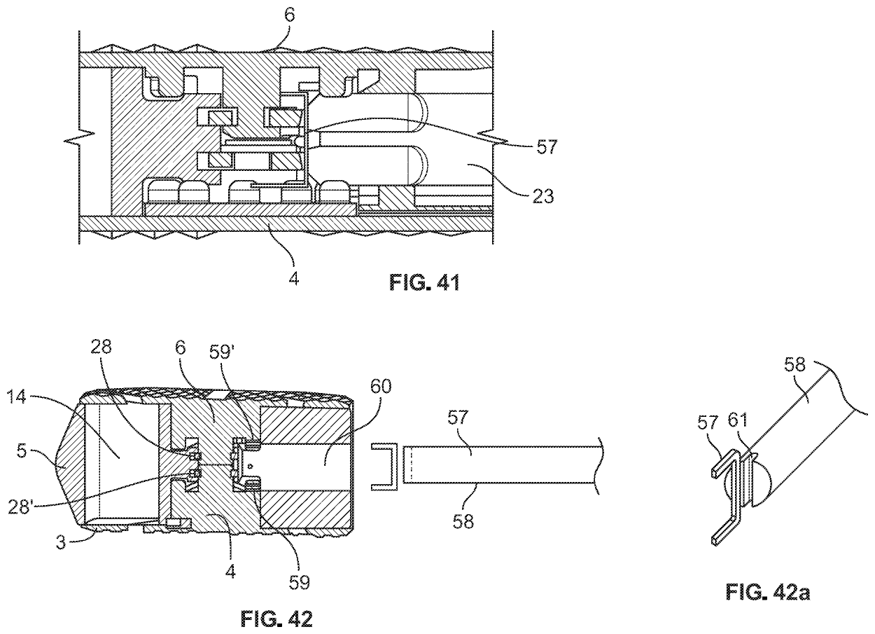

The head may also include locking mechanisms or members to hold the rotating blades in a deployed state or implanted configuration and prevent any further rotation of the blades. FIG. 41 shows an illustration of a locking member, such as the illustrated staple like locking feature 57, that spans across the top rotating blade 6 and the bottom rotating blade 4 once the rotating drive shaft 23 is removed. FIG. 42 shows a cross-section of the head 3 with the bone graft cavity 14, the top rotating blade 6 and its snap ring 28 to hold it in place into the main body 5. The bottom rotating blade 4 has snap ring 28′. Cavity 60 houses the rotating drive shaft 23 not shown. There is a locking hole 59 on the bottom rotating blade 4 and a counterpart locking hole 59′ on the top rotating blade 6. It is understood that multiple locking holes 59 & 59′ can be machined within each rotating blade to accommodate various angulation of the rotating blades as can be seen in FIG. 5, FIG. 20, and FIG. 23. Additionally, the locking member, such as staple like locking feature 57, may be an extension of the drive shaft. In such an embodiment, the locking feature 57 and/or drive shaft may be active to insert and release the locking member so that it engages the head 3 and locks the blades in place. The locking feature 57 can be activated or deployed by a trigger mechanism, plunger, or slide.

Locking staple 57 is shown prior to insertion on FIG. 42 with its inserter tool 58 and to facilitate the insertion. In FIG. 42a, the locking staple 57 has receptacle groove 61 to hold the staple in place during insertion. The receptable groove 61 and locking staple form a tight fit but not tight enough that it could not be removed. Other means to hold the locking staple 57 onto its inserter tool 58 can also be used as it is known in the art.

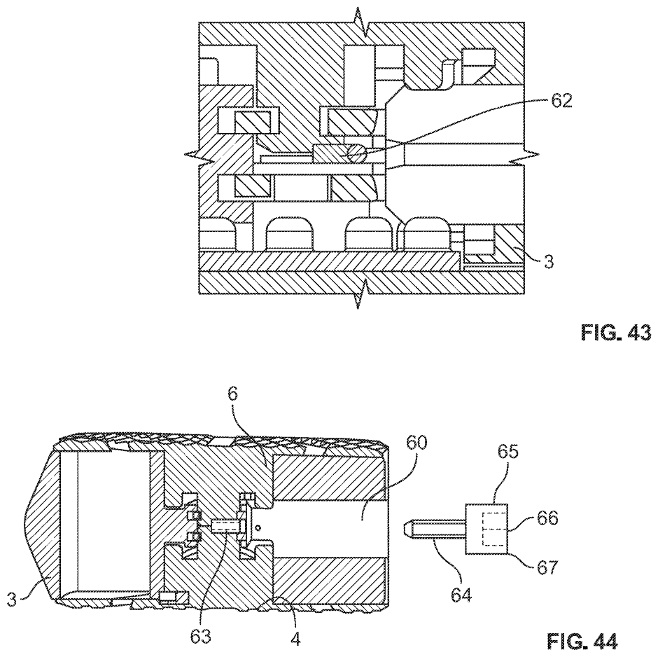

FIG. 43 illustrates another embodiment of a locking feature that includes a tapped hole 62 cut-out of the head 3.

FIG. 44 shows across-section of the head 3 with the top rotating blade 6, the bottom rotating blade 4 and the cavity 60. In addition, the tapped hole 63 is half tapped into top rotating blade and half tapped into bottom rotating blade. When both blades are in the deployed position, the halves of the tapped hole 63 are aligned, and it is possible to screw a locking socket head cap screw 67 which has the threaded portion 64, the screw head 65 and a recess 66 for accommodating a driver. For example, a hexagon recess for a socket head driver (not shown).

FIG. 47 illustrates another embodiment of a locking mechanism having an insert locking device 68, which has a distal section 69 with the same end shaped of the circumferential gear 25 of the driving shaft previously described and a square proximal section 70 section.

FIG. 46 shows a cross-section view illustrating that the insert locking device 68 has the tapped hole 71 at the proximal end to accommodate an inserter not shown that is screwed into that section for ease of insertion.

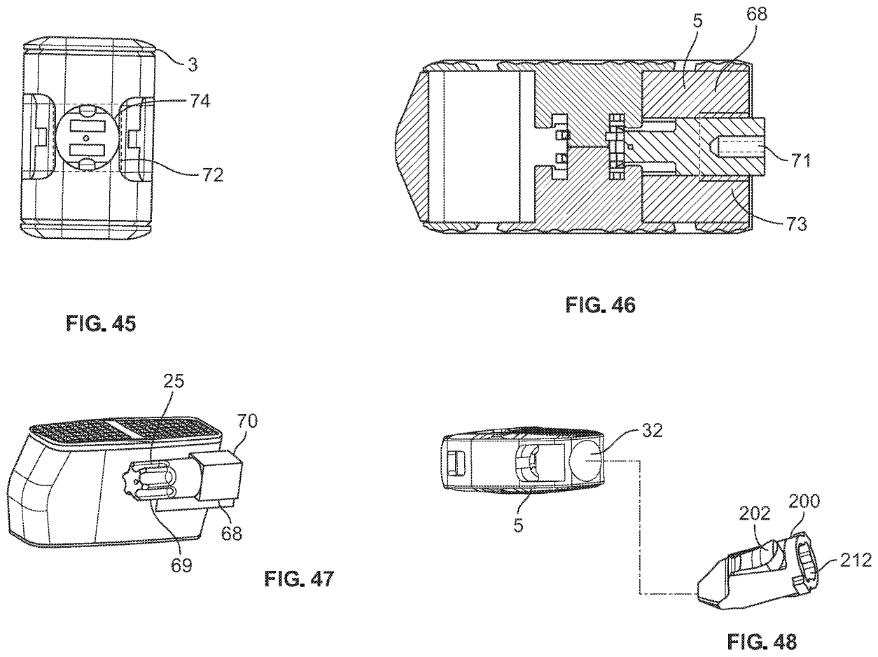

Referring to FIG. 45, the head 3 includes a square machined section 72 having a depth 73. The machined section 72 prevents the insert locking device 68 from rotating once the locking device is fully inserted inside the cavity 74 of the body 5. This prevents the rotating blade from further movement to lock the blades in the deployed/implanted configuration.

It is also understood that other locking mechanisms can be adapted to the head to lock the rotating blades such as using a keyway instead of the square design previously described. Or a split dowel pin that span across the two gearwheel or spring-loaded ball-nose pins that are located at the desired location to engage onto corresponding dimples once the blades are in the desired deployed position for example.

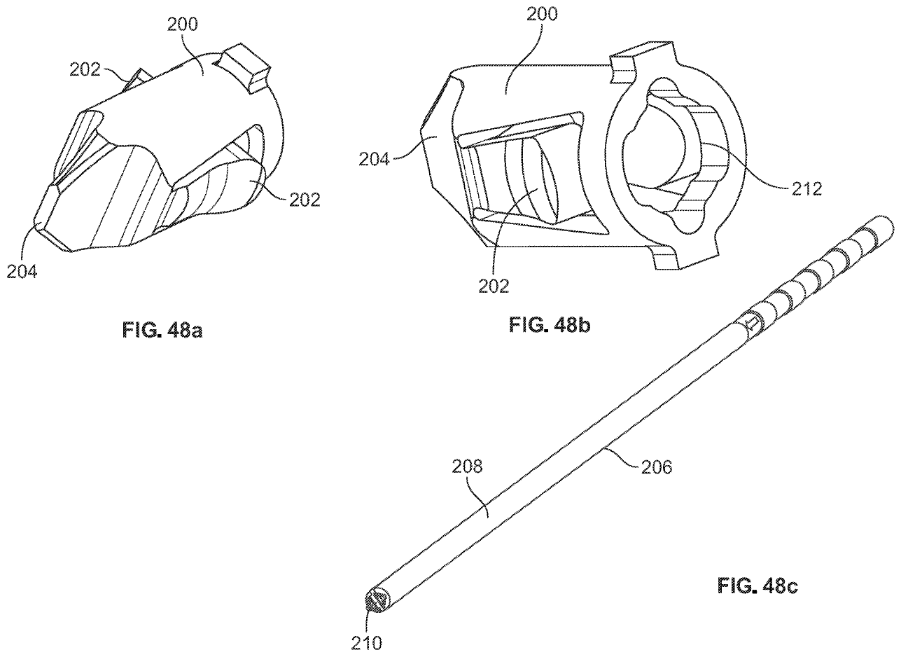

FIGS. 48-48c illustrate another embodiment of a locking device in the form of a locking pin 200. The locking pin 200 is inserted in the back of the body 5 in opening 32. The locking pin 200 includes a snap 202 that engages in the body 5 to securely lock the top and bottom blades 4 and 6 in place and prevent or reduce rotation. Referring to FIGS. 48a and 48b, locking pin 200 includes a locking wedge 204 that is configured to be positioned into the gears 26a, 26b (FIG. 9a) of the top and bottom blades 6a, 4a. The locking device may include an inserter 206 (FIG. 48c). The distal end 208 of inserter 206 includes a releasable holder 210 that is sized and shaped to be inserted into a bore 212 at the proximal end 214 of the locking pin 200.

After the head of the device has been placed in the disc space, the drive shaft 23 is removed from the deployment device 10. Referring to FIGS. 3b and 3d, the collar 145 is engaged with the handle 12 via threads. Optionally, the handle 12 has a safety lock in the form a screw 171. If a screw 171 is present, the screw is backed out and the collar 145 is unthreaded from the handle. This allows the shaft 23 to be removed from the deployment device 10.

After the head of the device has been placed in the disc space, the drive shaft 23 is removed from the deployment device 10. Referring to FIGS. 3b and 3d, the collar 145 is engaged with the handle 12 via threads. Optionally, the handle has a safety lock in the form a screw 171. If a screw 171 is present, the screw is backed out and the collar 145 is unthreaded from the handle. This allows the shaft 23 to be removed from the deployment device 10. With the drive shaft 23 removed, the inserter 206 carrying the locking pin 200 is inserted through the deployment device 10 and the locking pin is placed into bore 32 of the head 5. The wedge engages the gears and the snaps engage the head to hold the locking pin in place. Optionally, the locking pin may be keyed to the bore 32.

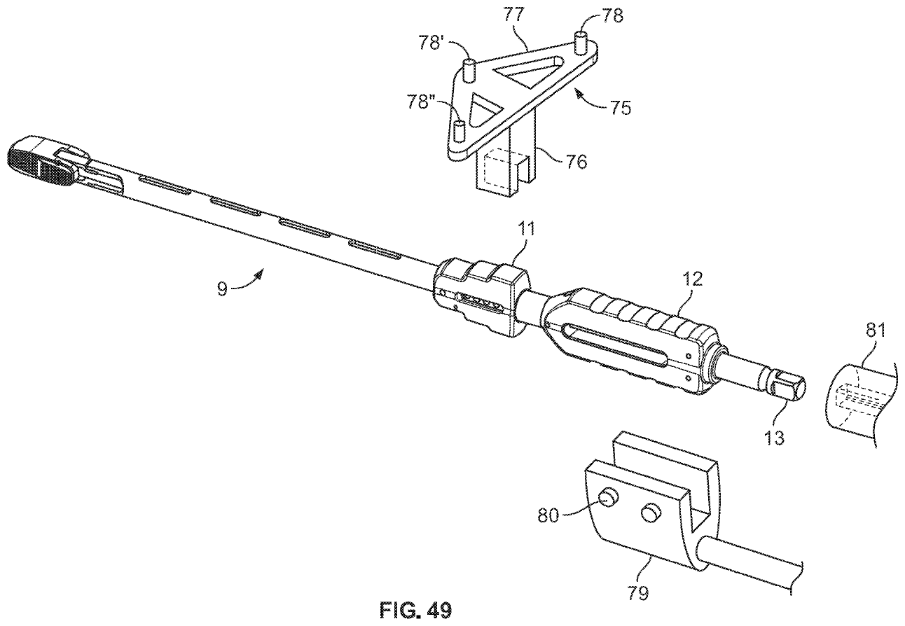

FIG. 49 shows features to accommodate robotic and/or motion control of the discectomy instrument 9 with its head 3 via motion control system for navigation of the instrument and implant during surgery. Some embodiments may include a triangulation guide assembly 75 that may be mounted on the controlling section 11 and is made of a post 76, a horizontal platform 77 and three triangulation reference posts 78, 78′ and 78″. An additional mounting guide 79 may be provided to be affixed to handle 12 and once mounted, could be secure via a pair of screws 80 as shown. An interface 81 would be mounted at the proximal end of discectomy instrument 9 with its the interface shaped accommodate the square interface 13 that would drive the rotating blades when required during the surgery.

Now it is understood that feature like the triangulation guide assembly 75, the mounting guide 79 and the interface 81 could have other shapes and different coupling to the controlling element of the control system used for spinal surgery as it is known in the art.

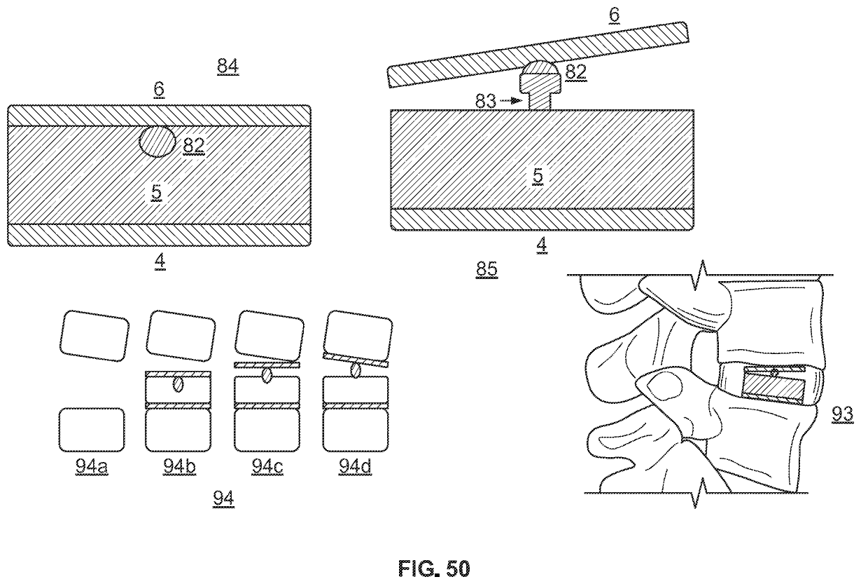

FIG. 50 shows two perspectives of a head; one in a non-expanded position 84 and one in the expanded position 85. The position of the ball and socket joint 82 within the shaver head body 5 can be seen in the non-expanded position 84.

The expanding member 83 can be seen housing the ball and socket joint 82, allowing the top blade 6 to tilt freely to accommodate the patient's anatomical lordotic angle 93. After the interface 13 is switched from rotation mode to expansion mode, turning of the interface 13 causes the extending member 83 to elevate the top blade 6. The introduction of the head/implant into the disc space and a step by step view of the expansion process 94 shows the empty disc space 94a, the introduction of the non-expanded implant 94b, the initial expansion of the top blade prior to contacting the vertebral endplate 94c, and the vertebral endplate's contact with the top blade causing the blade pivot on the ball and socket joint and conform to the lordotic angle of the patient's vertebral segment 94d.

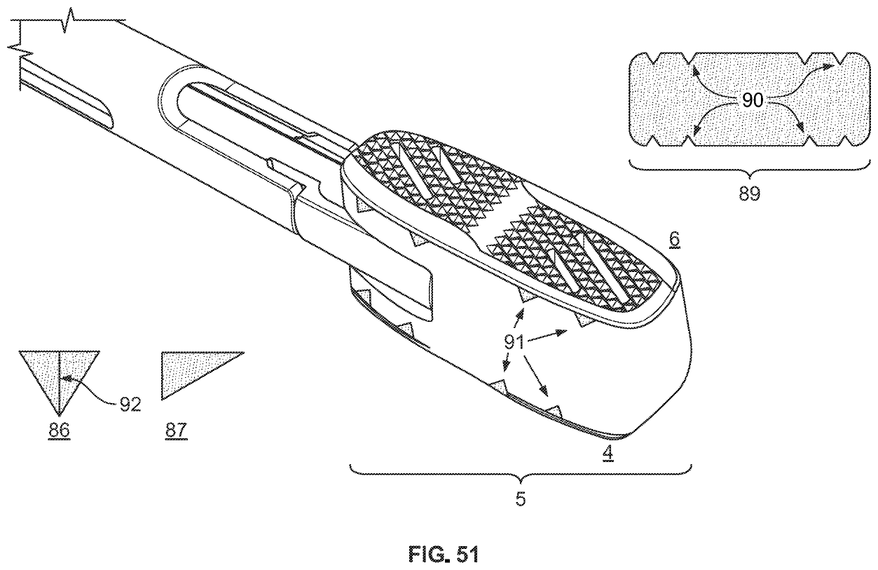

FIG. 51 shows an embodiment of the body 5 with multiple cutouts 90 to accommodate corresponding cutting fins 91 protruding from the top and bottom blades 6, 4. The cutouts 90 allow for passage of the cutting fins 91 during rotation of the blades, providing multi-directional dissection and fragmentation of the disc material during rotation. A front view 86 and side view 87 of the cutting fins are shown and depict a sharp cutting edge 92 on the front side of the cutting fins 91.

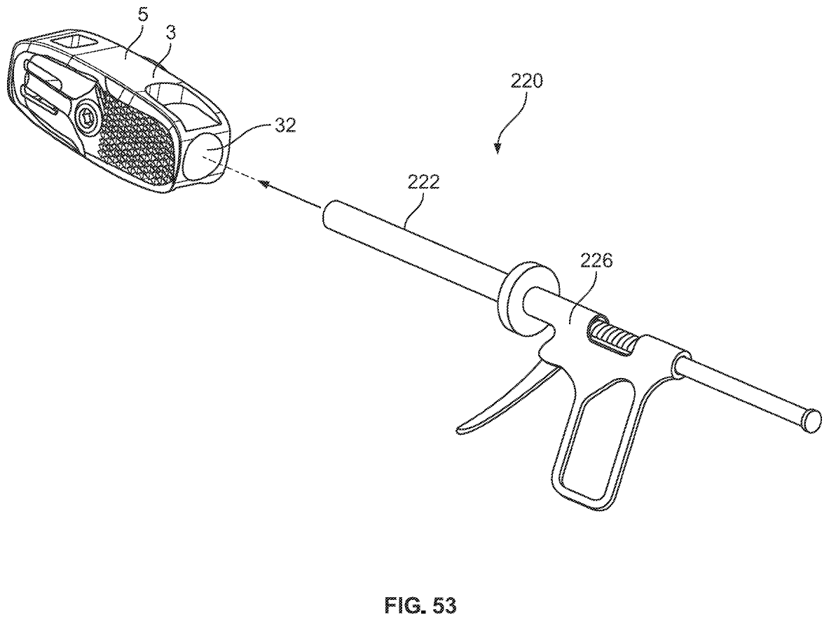

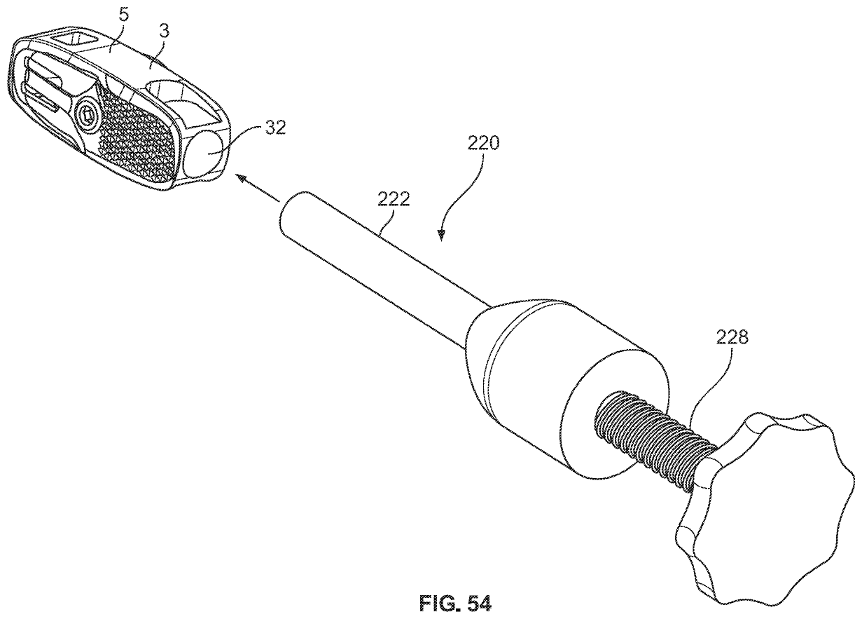

Turning to FIGS. 52-54, this figures illustrate the insertion of insert bone graft into a head 3. The bone graft delivery system 220 includes a funnel 222 and a bone graft deployment mechanism 224 (FIGS. 52), 226 (FIG. 53) or 228 (FIG. 54). Turning FIG. 52, funnel 222 is inserted into hole 32 in body 5. In some embodiments, hole 32 is the same hole in which the drive mechanism is positioned. Once the funnel 222 is inserted, bone graft material is placed into the funnel. Alternatively, the funnel could be pre-loaded with bone graft material.

Plunger 224 is inserted into the funnel 222 to push the bone graph material into body 5. The funnel and plunger can be used to delivery bone graph material after the head 3 has been deployed into the disc space. For example, the funnel and plunger can be adapted for insertion through the deployment device 10 after the shaft 23 has been removed.

FIG. 53 illustrates using a funnel and a pistol grip rachet mechanism 226 to deliver bone graft material into the head 3. FIG. 54 illustrates using the funnel 222 and a screw advancing mechanism 228 to deliver the bone graft material into head 3. The advancing screw mechanism may provide greater force to the delivery of the bone graft material, resulting in better compaction the material in the head 3.

Aspect 1. A device for treating a intervertebral disc space, comprising: a deployment device having a proximal end and a distal end; a head releasably attached the distal end of the deployment device, the head comprising: a body having a top portion and a bottom portion; a top rotatable blade associated with the top portion of the body and rotatable relative to the body; a bottom rotatable blade associated with the bottom portion of the body and rotatable relative to the body; and a drive mechanism to rotate the top and bottom rotatable blades.

Aspect 2. The device of Aspect 1, wherein the head has an initial configuration and wherein the top and bottom rotatable blades are aligned with the body.

Aspect 3. The device of any one of Aspects 1 and 2, wherein the head has a deployed configuration wherein the top and bottom rotatable blades are misaligned with the body.

Aspect 4. The device of Aspect 3, wherein the top and bottom rotatable blades are misaligned with each other.

Aspect 5. The device of any one of Aspects 3 and 4, wherein the blades are locked in the deployed configuration.

Aspect 6. The device of any one of Aspects 3-5, furthering including a locking mechanism to lock the top and bottom rotatable blades in the deployed configuration.

Aspect 7. The device of any of Aspects 1-6, wherein the top and bottom blades rotate about a transverse axis that is perpendicular to a longitudinal axis of the body.

Aspect 8. The device of any of Aspects 1-7, wherein the top and bottom rotatable blades are configured to rotate in opposite directions.

Aspect 9. The device of any one of Aspects 1-8, wherein at least one of the top and bottom rotatable blades have a tissue disruption element.

Aspect 10. The device of Aspect 9, wherein the tissue disruption element comprises a cutting edge extending at least partially along one side of respective blade.

Aspect 11. The device of any one of Aspects 9 and 10, wherein the tissue disruption element comprises a rasp-like surface having projections and recesses.

Aspect 12. The device of any one of Aspect 1-11, furthering including one or more expanding plates attachable to one of the top rotatable blade, the bottom rotatable blade or both.

Aspect 13. The device of any one of Aspects 1-12, wherein the body comprises a top protection lip and a bottom protection lip at the distal end of the body, wherein each of the protection lips comprise a shoulder and a recess, and wherein distal ends of the top and bottom blades are located in the respective recess of the protection lips.

Aspect 14. The device of any one of Aspects 1-13, wherein the deployment device has a releasable attachment mechanism attached to the head.

Aspect 15. The device of Aspect 14, wherein the releasable attachment mechanism comprises a pair of prongs that grasp the head.

Aspect 16. The device of Aspect 15, wherein the prongs have an initial grasping configuration and a releasing configuration, wherein in the releasing configuration the prong move apart from one another to release the head.

Aspect 17. The device of any one of Aspects 1-16, wherein the drive mechanism comprises a drive shaft having gear teeth that engage gear teeth of the top and bottom blades.

Aspect 18. The device of Aspect 16, wherein the drive mechanism comprises a miter gear.

Aspect 19. The device of any one of Aspects 1-13, wherein the deployment device comprises a shaft assembly including an outer sleeve, a grabbing sleeve and a drive shaft.

Aspect 20. The device of Aspect 19, wherein the grabbing sleeve and drive shaft are positioned in the outer sleeve.

Aspect 21. The device of Aspect 20, wherein the grabbing sleeve includes a pair of prongs at the distal end of thereof, the prongs having an initial grasping configuration and a releasing configuration, wherein in the releasing configuration the prong move apart from one another to release the head, and wherein the outer sleeve holds the prongs in the initial grasping configuration and the outer sleeve is movable to allow the prongs to move to the releasing configuration.

Aspect 22. An intravertebral disc treatment device, comprising: a head configured to be releasably attached a delivery device, the head comprising: a body having a top portion and a bottom portion; a top rotatable blade associated with the top portion of the body and rotatable relative to the body; a bottom rotatable blade associated with the bottom portion of the body and rotatable relative to the body; and recesses in the body configured to be releasably grasped by a deployment device.

Aspect 23. The device of Aspect 22, wherein latches are located in the recesses, wherein the latches are configured to be grasped by grasping member of the deployment device.

Aspect 24. The device of any one of Aspects 22 and 23, wherein the head has an initial configuration and wherein the top and bottom rotatable blades are aligned with the body.

Aspect 25. The device of any one of Aspects 22-24, wherein the head has a deployed configuration wherein the top and bottom rotatable blades are misaligned with the body.

Aspect 26. The device of Aspect 25, wherein the top and bottom rotatable blades are misaligned with each other.

Aspect 27. The device of any one of Aspects 25 and 26, wherein the blades are locked in the deployed configuration.

Aspect 28. The device of any one of Aspects 25-27, furthering including a locking mechanism to lock the top and bottom rotatable blades in the deployed configuration.

Aspect 29. The device of any of Aspects 22-28, wherein the top and bottom blades rotate about a transverse axis that is perpendicular to a longitudinal axis of the body.

Aspect 30. The device of any of Aspects 22-29, wherein the top and bottom rotatable blades are configured to rotate in opposite directions.

Aspect 31. The device of any one of Aspects 22-30, wherein at least one of the top and bottom rotatable blades have a tissue disruption element.

Aspect 32. The device of Aspect 31, wherein the tissue disruption element comprises a cutting edge extending at least partially along one side of respective blade.

Aspect 33. The device of any one of Aspects 31 and 32, wherein the tissue disruption element comprises a rasp-like surface having projections and recesses.

Aspect 34. The device of any one of Aspect 22-33, furthering including one or more expanding plates attachable to one of the top rotatable blade, the bottom rotatable blade or both.

Aspect 35. The device of any one of Aspects 22-34, wherein the body comprises a top protection lip and a bottom protection lip at a distal end of the body, wherein each of the protection lips comprise a shoulder and a recess, and wherein distal ends of the top and bottom blades are located in the respective recess of the protection lips.

It is understood that the foregoing merely illustrates the principles of the various embodiments of the systems, device and methods disclosed herein. Various modifications and alterations to the described embodiments will be apparent to those skilled in the art in view of the teachings herein.

What is claimed is:

1. A device for treating a intervertebral disc space, comprising:
a deployment device having a proximal end and a distal end;
a head releasably attached the distal end of the deployment device, the head comprising:
a body having a top portion and a bottom portion;
a top rotatable blade associated with the top portion of the body and rotatable relative to the body;
a bottom rotatable blade associated with the bottom portion of the body and rotatable relative to the body; and
a drive mechanism to rotate the top and bottom rotatable blades.

2. The device of claim 1, wherein the head has an initial configuration and wherein the top and bottom rotatable blades are aligned with the body.

3. The device of claim 1, wherein the head has a deployed configuration wherein the top and bottom rotatable blades are misaligned with the body. (original) The device of claim 3, wherein the top and bottom rotatable blades are misaligned with each other.

5. The device of claim 3, wherein the blades are locked in the deployed configuration.

6. The device of claim 3, furthering including a locking mechanism to lock the top and bottom rotatable blades in the deployed configuration.

7. The device of claim 1, wherein the top and bottom blades rotate about a transverse axis that is perpendicular to a longitudinal axis of the body.

8. The device of claim 1, any of claims 1-7, wherein the top and bottom rotatable blades are configured to rotate in opposite directions.

9. The device of claim 1, wherein at least one of the top and bottom rotatable blades have a tissue disruption element.

10. The device of claim 9, wherein the tissue disruption element comprises a cutting edge extending at least partially along one side of respective blade.

11. The device of claim 9, wherein the tissue disruption element comprises a rasp-like surface having projections and recesses.

12. The device of claim 1, furthering including one or more expanding plates attachable to one of the top rotatable blade, the bottom rotatable blade or both.

13. The device of claim 1, wherein the body comprises a top protection lip and a bottom protection lip at the distal end of the body, wherein each of the protection lips comprise a shoulder and a recess, and wherein distal ends of the top and bottom blades are located in the respective recess of the protection lips.

14. The device of claim 1, wherein the deployment device has a releasable attachment mechanism attached to the head.

15. The device of claim 14, wherein the releasable attachment mechanism comprises a pair of prongs that grasp the head.

16. The device of claim 15, wherein the prongs have an initial grasping configuration and a releasing configuration, wherein in the releasing configuration the prong move apart from one another to release the head.

17. The device of claim 1, wherein the drive mechanism comprises a drive shaft having gear teeth that engage gear teeth of the top and bottom blades.

18. (canceled)

19. The device of claim 1, wherein the deployment device comprises a shaft assembly including an outer sleeve, a grabbing sleeve and a drive shaft.

20. The device of claim 19, wherein the grabbing sleeve and drive shaft are positioned in the outer sleeve.

21. (canceled)

22. An intravertebral disc treatment device, comprising:
a head configured to be releasably attached a delivery device, the head comprising:
a body having a top portion and a bottom portion;
a top rotatable blade associated with the top portion of the body and rotatable relative to the body;
a bottom rotatable blade associated with the bottom portion of the body and rotatable relative to the body; and recesses in the body configured to be releasably grasped by a deployment device.

23.-35. (canceled)
